(19) United States
(12) Patent Application Publication
(10) Pub. No.: US 2024/0189316 A1
(43) Pub. Date: Jun. 13, 2024

(54) DEUTERATED ANALOGS OF ETIFOXINE AND METHODS OF ADMINISTRATION WITHOUT AUTOINDUCTION OF METABOLISM

(71) Applicant: Gaba Therapeutics Inc., Newport Beach, CA (US)

(72) Inventors: Olivier Dasse, Foothills Ranch, CA (US); Mario Saltarelli, Tiburon, CA (US)

(21) Appl. No.: 18/493,488

(22) Filed: Oct. 24, 2023

Related U.S. Application Data
(60) Provisional application No. 63/380,737, filed on Oct. 24, 2022.

(51) Int. Cl. A61K 31/536; A61K 9/00; A61P 25/22

(52) U.S. Cl. CPC A61K 31/536; A61K 9/0053; A61P 25/22

(57) ABSTRACT

The present invention relates to deuterated etifoxine compositions and methods for administration without autoinduction of metabolism in the treatment of a variety of diseases. disorders, or conditions.

### FIELD OF THE INVENTION

The present disclosure relates to deuterated etifoxine compositions and methods of administering deuterated etifoxine without autoinduction of metabolism. The compositions and methods disclosed herein are useful for treating a wide variety of conditions, e.g., treating anxiety, and result in lower dosages and improved dosing frequency of deuterated etifoxine.

### BACKGROUND OF THE INVENTION

Absorption, distribution, metabolism and excretion (ADME) properties of drugs are critical characteristics of any drug and can mean the difference between a safe and effective drug on the one hand, and a clinical and commercial failure, on the other hand. While recent advances in drug formulation technologies (and drug conjugates or prodrugs) have offered some ability to improve ADME in limited cases, underlying ADME problems are still a major cause of the failure of drugs in clinical trials. A common ADME issue with currently approved drugs and drug candidates is rapid metabolism. A drug candidate that otherwise is highly efficacious in in vitro and preclinical testing, can be metabolized too quickly and cleared from the body giving little to no pharmacological effect. “Band Aid” efforts to overcome fast metabolism include dosing at very high levels or dosing very frequently. Both of these solutions to rapid metabolism are fraught with problems, including increasing the side effects of drugs to maintain therapeutic drug levels, increasing exposure to metabolites that may be toxic or induce increased metabolism of concomitant medications or its own metabolism, and decreasing patient compliance due to frequent dosing during the day.

In limited cases, metabolic inhibitors have been used to improve the characteristics of a particular drug (see Kempf. D. et al. Antimicrobial Agents and Chemotherapy, 41(3), p.654 (1997); Wang. L. et al. Clinical Pharmacology and Therapeutics, 56(6 Pt.1), p.659 (1994). However, this strategy is not widely used, and can lead to serious unwanted side effects, and undesired drug-drug interactions.

Optimization of drug structure by chemists usually involves an iterative process of structure modification to improve biological activity and/or metabolic properties. However, a better metabolic profile often comes at the expense of biological potency and efficacy, due to the significant structural modifications of a desired pharmacophore structure needed to stop or slow biological degradation processes. A potential strategy for improving the metabolic profile of a drug, without substantially altering the biological potency and efficacy, is to replace (substitute) one or more hydrogen atoms with deuterium, thus slowing cytochrome P450 mediated metabolism. Deuterium is an isotope of hydrogen that contains an additional neutron in its nucleus, and is safe, stable and nonradioactive. Due to the increased mass of deuterium as compared to hydrogen, the bond between carbon and deuterium has a higher energy (stronger) as compared to the bond between hydrogen and carbon, and can reduce metabolic reaction rates. The reduced metabolic reaction rate can favorably impact a molecule's ADME properties, giving improved potency, efficacy, safety and tolerability. Other physical characteristics of deuterium are essentially identical to hydrogen, and would not be expected to have a biological impact on a molecule with deuterium replacement.

In nearly four decades, only a small number of drugs have been approved that employ deuterium substitution to improve metabolism (see Blake, M. et al. J. Pharm. Sci., 64, p.367 (1975); Foster, A. Adv. Drug Res., 14, p.1 (1985); Kushner, D. et al. Can. J. Physiol. Pharmacol., p.79 (1999); Fisher M. et al. Curr. Opin. Drug Discov. Devel., 9, p.101 (2006)). The result of deuterium replacement of hydrogen on metabolic rate, however, has not been predictable and has led to variable results. In some cases, the deuterated compounds had a decreased metabolic clearance in vivo, however for others, there was no change in the clearance rate, and yet others unexpectedly showed an increase in metabolic clearance rate. This variability has led ADME experts to question or reject deuterium replacement as a strategic drug design modification for reducing metabolic rate (see Foster and Fisher).

Even when a site and position of metabolism is known, deuterium replacement does not have a predictable effect on the metabolic rate. It is only by preparation of the specific deuterium substituted drug (candidate) and testing that one can determine the extent of change in metabolic rate. See Fukuto, J. et al. J. Med. Chem., 34(9), p.2871 (1991). Many, if not most, drug candidates have multiple sites where metabolism is possible, however, this is unique to each drug molecule, thus making deuterium replacement a different study for its effect on each candidate. See Harbeson, L. and Tung, R. Medchem News, 2, p.8 (2014) and references therein. There are several examples of drug candidates where deuterium substitution of hydrogen has led to an enhanced metabolic rate and/or metabolic switching, or no in vivo change of the molecule's profile even after metabolic slowing. Harbeson et al. reveal that selective deuteration of paroxetine at predicted metabolically labile positions actually produced analogs which demonstrated increased metabolism in vivo (Scott L. Harbeson and Roger D. Tung, Deuterium in Drug Discovery and Development, 46 annual report in medicinal chemistry, 403-417 (2011)). Furthermore, Miwa reports that deuteration of metabolically labile sites may lead to the potentiation (or switching) of alternative metabolic pathways, with then undetermined consequences (Miwa, G., Lu, A., Kinetic Isotope Effects and ‘Metabolic Switching’ in Cytochrome P450-Catalyzed Reactions, 7 Bioessays, 215-19 (1987)). Phentermine has been deuterated to decrease its metabolic rate, however replacement of N,N-dimethyl hydrogens with deuterium resulted in no change observed (Allan B. Foster, “Deuterium Isotope Effects in the Metabolism of Drugs and Xenobiotics: Implications for Drug Design”, Advances in Drug Research, (14), 1-40 (1985)). Similarly, deuteration of metabolically active sites of tramadol led to no increase in duration of effect (Shao et. al. , “Derivatives of Tramadol for Increased Duration of Effect”, Bioorganic and Medicinal Chemistry Letters, (16),691-94 (2006)).

Etifoxine [6-chloro-2-(ethylamino)-4-methyl-4-phenyl-4H-3,1-benzoxazine] was originally disclosed in U.S. Pat. No. 3,725,404 by Hoffmann, I et al. Etifoxine has been shown to be an effective, acute acting, anxiolytic agent in humans with minimal sedative and ataxic side effects. Stein, D., Adv. Ther. 32(1), p.57 (2015); Nguyen, N. et al., Hum. Psychopharm. 21, p.139 (2006); Micallef, J., Fundam. Clin. Pharmacol., 15(3), p.209 (2001).

The hydrochloride salt of etifoxine [6-chloro-2-(ethylamino)-4-methyl-4- phenyl-4H-3,1-benzoxazine] is known as Stresam™ and is sold mainly in France and in a limited number of other markets around the world for the treatment of anxiety (specifically, anxiety with somatic manifestations). The short half-life of etifoxine in humans (4-6 hours) is a significant limitation in its use. The recommended dosing schedule for etifoxine is three times a day (or a higher dose, twice a day). This schedule can be quite inconvenient to the patient and can contribute to dosing noncompliance and reduced efficacy. See Santana, L. et al, Patient Preference and Adherence, 5, p.427 (2011). Studies also show a significant individual variability of the pharmacokinetic parameters especially in the dose Cₘₐₓ relationship (see etifoxine package insert information, Lundbeck Argentina SA). Inter and intra-patient variability is largely based on differences in drug metabolic capacity. Reducing inter- and intra-patient variability is desirable as it hampers optimal therapy. Poor metabolizers may be at higher risk of off-targets side-effects due to higher drug levels. Excessive metabolizers may not get relief from insufficient efficacy due to excessively diminished drug levels. (see Wilkinson, G. The New England Journal of Medicine (352), 2211-21 (2005). Enhancing the metabolic stability of etifoxine will reduce inter- and intra-patient variability as metabolic capacity becomes less of a determining factor in the drugs ADME.

Deuterated etifoxine for treatment of anxiety was previously disclosed in U.S. Pat. Nos. 10,080,755 and 10,736,901, both entitled “Deuterated Analogs of Etifoxine, Their Derivatives and Uses Thereof,” to Olivier Dasse. This patent disclosed that deuterated etifoxine hydrochloride administered to rats at 50 mg/kg achieved a significantly higher AUC and Cₘₐₓ compared to non-deuterated etifoxine hydrochloride. The AUC₀₋₁₂ and Cₘₐₓ of the hydrochloride salt of the compound described in example 1 are 2.5 times and 1.7 times greater than etifoxine hydrochloride. These results indicate decreased pre-systemic metabolism resulting in higher bioavailability of the unchanged drug. Decreasing pre-systemic metabolism may result in lesser inter and intra-dose variability. Increasing drug exposure may result in reduced dosing frequency, as minimal drug therapeutic levels may be achieved for longer period of times. Increasing drug exposure also allows for dose lowering, resulting in less potential adverse events as similar drug plasma levels can be achieved with a lower dose.

Etifoxine exhibits an auto-induction effect whereby continued dosing over time results in an induction of CYP enzyme(s) that are responsible for the metabolism of these agents. The phenomenon of auto-induction cannot be observed from single dose studies and requires carefully monitoring the effects of administration of the drug over a multi-day period. Where auto-induction occurs, an increased dose of drug may be needed over time to counteract the effect of increased metabolism. This becomes increasingly important in the development of pharmaceutical products for treating chronic conditions, where multi-day dosing and long term continuous treatment is required. Consequently, despite the desirable and beneficial effects of etifoxine, there is a continuing need to develop treatment regimens that improve drug bioavailability over time for chronic conditions.

### SUMMARY OF THE INVENTION

Briefly, this invention is generally directed to methods for treating anxiety and other disorders in a subject in need thereof, wherein the compound is a compound of formula (I):

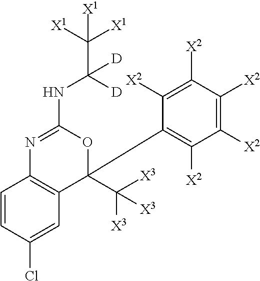

including pharmaceutically acceptable salts, solvates, and prodrugs thereof, wherein each X¹, X², X³ are independently selected from hydrogen or deuterium, wherein said compound is administered at a dose and frequency that is effective for treatment of a disease, disorder, or condition without autoinducing metabolism of the compound. The autoinduction of metabolism may be quantified by in an ARCₘₐₓ and ARAUC₀₋₁₂ over a period of 7 days that is less than 1.0. Accordingly, the present invention comprising administering a drug at a dosage and frequency that results in an ARCₘₐₓ and ARAUC₀₋₁₂ over a period of 7 days that is less than 1.0. Levels of ARCₘₐₓ and ARAUC₀₋₁₂ over a period of 7 days that are greater than or equal to 1.0 indicate a lack of autoinduction.

According to specific embodiments the dosing frequency may be varied to include QID (four-times daily), TID (three-times daily), BID (twice-daily), or QD (once daily) depending on the dose. In one aspect, the dose may include 100 mg or less. The dose may further range between 25 to 100 mg, including any value in between. In certain instances, the dose may range between 50 mg to 100 mg. In one embodiment of the invention, the dose is 60 mg administered QD (once-daily).

The methods of treatment of anxiety may include one or more specific kinds of anxiety including but not limited to one or more of panic disorder without agoraphobia, panic disorder with agoraphobia, agoraphobia without history of panic disorder, specific phobia, social phobia, obsessive compulsive disorder, posttraumatic stress disorder, acute stress disorder, generalized anxiety disorder, social anxiety disorder, anxiety disorder due to a medical condition, substance induced anxiety disorder, and/or anxiety with somatic manifestations, anxiety with co-morbid depression, adjustment disorder with anxiety, separation anxiety, acute anxiety, selective mutism, medication-induced anxiety disorder.

The compound may be one or more of the compounds selected from Formula (I) above, including specifically 6-chloro-N-(ethyl-d5)-4-methyl-4-phenyl-4H-3,1-benzoxazin-2-amine, or a pharmaceutically acceptable salt thereof. It should be understood that the level of deuterium isotope exceeds the natural abundance of deuterium. For example, the compound may include deuterium in an abundance that is at least 3340 times greater than the natural abundance of deuterium. The composition in certain embodiments is a racemate. Other forms of the compound of Formula (I) may be used including the R-enantiomer or S-enantiomer, as well as mixtures of these enantiomers in various ratios such as 3:1 or 4:1 etc.

### BRIEF DESCRIPTION OF THE FIGURES

FIG. 1 is a chart showing Mean±SD Plasma Concentration-time Profile following 50 mg/kg GRX-917 and Etifoxine in Rats.

FIG. 2A Arithmetic Mean Plasma Concentrations of deuterated etifoxine Following Single Oral Doses, Day 1 (Linear Scale) in healthy human volunteers.

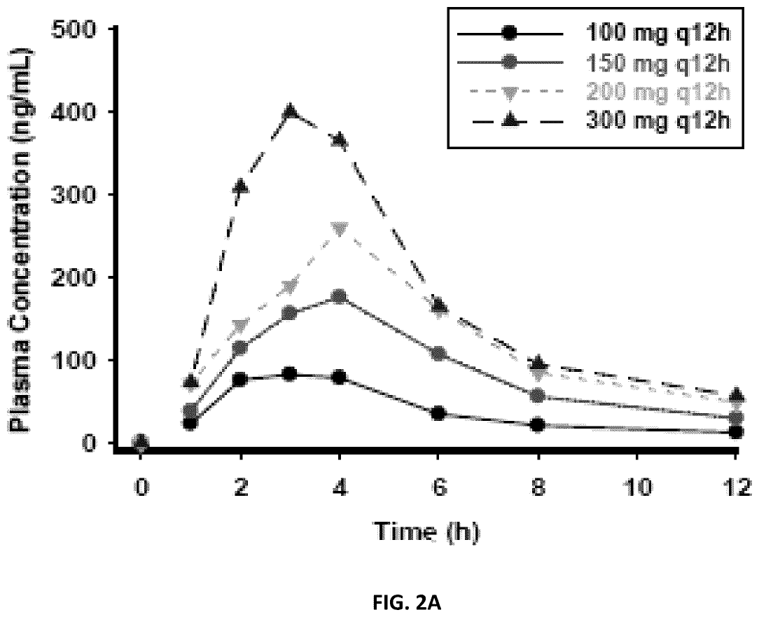

FIG. 2B Arithmetic Mean Plasma Concentrations of deuterated etifoxine Following Single Oral Doses, Day 1 (Semi-logarithmic Scale) in healthy human volunteers.

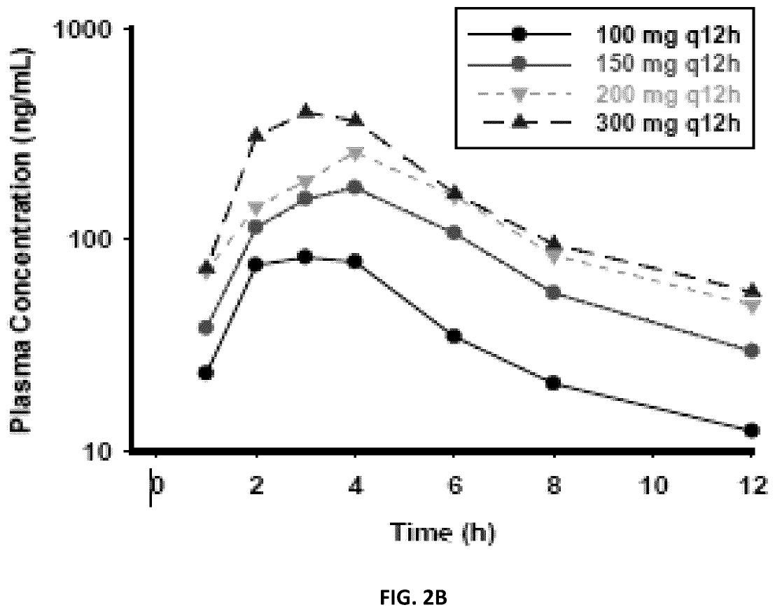

FIG. 3A Arithmetic Mean Plasma Concentrations of deuterated etifoxine Following Single Oral Doses of deuterated etifoxine Administered q12 h (bid) for 7 Days (Linear Scale) in healthy human volunteers.

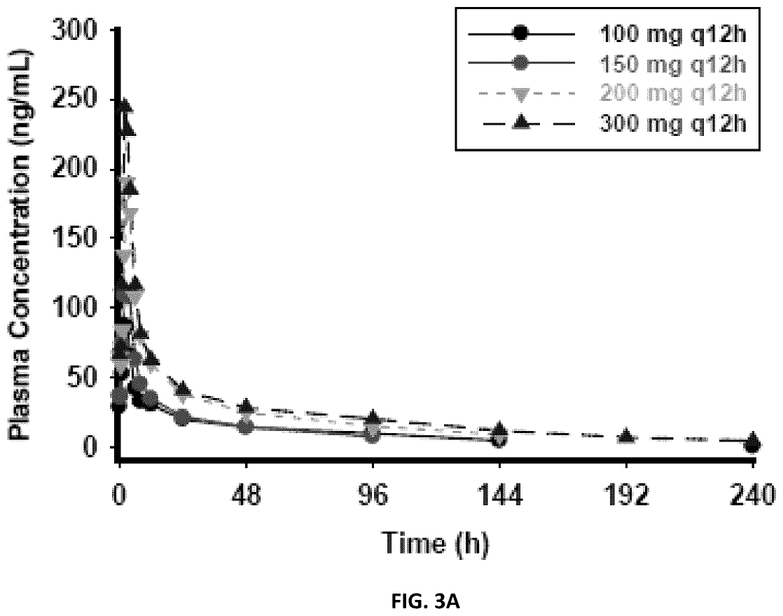

FIG. 3B Arithmetic Mean Plasma Concentrations of deuterated etifoxine Following Single Oral Doses of deuterated etifoxine Administered q12 h (bid) for 7 Days (Semi-logarithmic Scale) in healthy human volunteers.

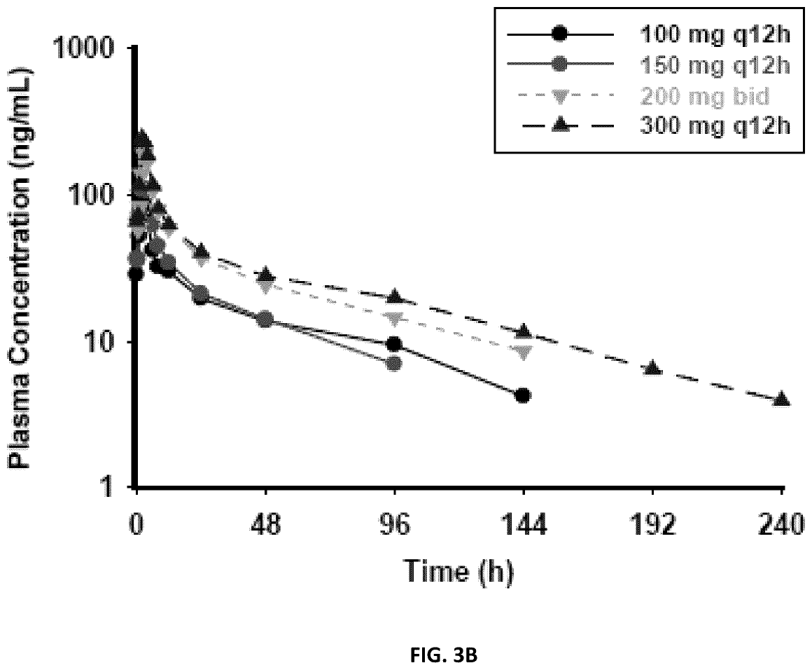

### DETAILED DESCRIPTION

The present invention is directed to the therapeutic use of deuterated analogs of etifoxine in dosage regimens that are suitable for multi-day administration, such as in the treatment of chronic conditions such as chronic anxiety.

Etifoxine has been studied extensively preclinically and has demonstrated efficacy in many animal models of CNS and mental disorders such as anxiety, pain, inflammation, neurodegeneration, inflammatory pain, nerve damage, Multiple Sclerosis, alcohol withdrawal, epilepsy and light-induced lesions of the retina. Verleye, M. et al., Pharmacol. Biochem. Behav., 82(4), p. 712 (2005); Ugale, R. et al., Brain Res., 12, p. 193 (2007); Verleye, M. et al., Alcohol, 43(3), p. 197 (2009); Aouad, M. et al., Pain, 147(1-3), p. 54 (2009); Girard, C. et al., J. Neuroendocrinol., 24(1), p. 71 (2012); Zhou, X. et al., Mol. Med. Rep., 8(1), p. 75 (2013); Aouad. M. et al., Eur. J. Pain. 18(2), p. 258 (2014); Aouad. M. et al., Pain, 155(2), p. 408 (2014); Zhou, X. et al., Muscle Nerve, 50(2), p. 235 (2014): Dai, T. et al., J. Reconstr. Microsurg., 30(6), p. 381 (2014); Juif, P. et al., Neuropharmacology, 91, p. 117 (2015), Verleye, M et al. WO 2015113991.

Etifoxine has been described in scientific literature to act through allosteric modulation of the GABA.sub.A ion channel complex as well as increasing the levels of endogenous neurosteroids and neuroactive steroids. Verleye, M. et al., Neuroreport., 10(15), p. 3207 (1999): Verleye, M. et al., Neurosci. Lett., 301(3), p. 191 (2001), Hamon, A. et al., Neuropharmacology, 45(3), p. 293 (2003); Ugale, R. et al., Brain Res., 12, p. 193 (2007); Verleye, M. et al., Pharmacol. Biochem. Behav., 82(4), p. 712 (2005).

Neurosteroids and neuroactive steroids have demonstrated anti-inflammatory activity, for instance progesterone and allopregnanolone reduce both cytokines IL-1β and TNF-α in a model of TBI (see He, J. et al. Experimental Neurology, 189, p. 404 (2004)). Furthermore, dehydroepiandosterone (DHEA), which is mainly synthesized in the adrenal glands, inhibits the synthesis of cytokines IL-6 and TNF (see Straub, R. Rheumatology, 39, p. 624 (1999). By increasing levels of neurosteroids and/or neuroactive steroids, it is thought that etifoxine may be effective in treating neuroinflammation, peripheral inflammation and a variety of inflammatory conditions.

Neurosteroids and neuroactive steroids have been shown to be neuroregenerative and neuroprotective in preclinical models, see Brinton, R. Nature Reviews Endocrinology 9, 241-250 (2013) and Borowicz, K, et. al. Frontiers in Endocrinology 2(50), P.1 (2011). Likewise, etifoxine has also demonstrated neuroregenerative and neuroprotective effects preclinically (Girard et. al. Journal of Neuroendocrinology 24, 71-81 (2011), Girard et. al. Clinical and Experimental Pharmacology and Physiology 36, 655-661(2009), Zhou et. al. Muscle Nerve. 50(2):235-43 (2014)).

### Definitions

Unless specifically noted otherwise herein, the definitions of the terms used are standard definitions used in the art of organic synthesis and pharmaceutical sciences.

The articles “a” and “an” are used herein to refer to one or more than one (i.e. to at least one) of the grammatical object of the article. By way of example, “an element” means one element or more than one element.

As used herein, the term “or” is generally employed in the sense as including “and/or” unless the context of the usage clearly indicates otherwise.

Where the plural form is used for compounds, salts and the like, this is taken to mean also a single compound, salt, or the like.

As used herein “solvate” refers to a complex of variable stoichiometry formed by a solute (e.g. a compound of formula (I) or a salt, ester or prodrug thereof) and a solvent. Such solvents for the purpose of the invention may not interfere with the biological activity of the solute. Examples of suitable solvents include water, methanol, ethanol and acetic acid. Generally the solvent used is a pharmaceutically acceptable solvent. Examples of suitable pharmaceutically acceptable solvents include water, ethanol and acetic acid. Generally the solvent used is water.

“Isomers” mean any compound with an identical molecular formula but having a difference in the nature or sequence of bonding or arrangement of the atoms in space. Examples of such isomers include, for example, E- and Z-isomers of double bonds, enantiomers, and diastereomers. Compounds of the present invention depicting a bond with a straight line, unless specifically noted otherwise, is intended to encompass a single isomer and/or both isomers and means any compound with an identical molecular formula but having a difference in the nature or sequence of bonding or arrangement of the atoms in space.

The term “GABA_{A} receptor” refers to a protein complex that detectably binds GABA and mediates a dose dependent alteration in chloride conductance and membrane polarization. Receptors comprising naturally-occurring mammalian (especially human or rat) GABA_{A} receptor subunits are generally preferred, although subunits may be modified provided that any modifications do not substantially inhibit the receptor's ability to bind GABA (i.e., at least 50% of the binding affinity of the receptor for GABA is retained). The binding affinity of a candidate GABA_{A} receptor for GABA may be evaluated using a standard ligand binding assay known in the art. There are a variety of GABA_{A} receptor subtypes that fall within the scope of the term “GABA_{A} receptor.” These subtypes include, but are not limited to α.sub. 1-6, β.sub. 1-3, γ.sub. 1-3, π, θ, ε, δ, and σ.sub. 1-3 receptor subtypes. GABA_{A} receptors may be obtained from a variety of sources, such as from preparations of rat cortex or from cells expressing cloned human GABA_{A} receptors. Particular subtypes may be readily prepared using standard techniques (e.g., by introducing mRNA encoding the desired subunits into a host cell).

As used herein, a “CNS disorder” is a disease or condition of the central nervous system that can be treated, prevented, managed or ameliorated with a compound or composition provided herein. Certain CNS disorders are responsive to GABA_{A} receptor modulation in a subject and some CNS disorders are responsive to increasing endogenous neurosteroids and neuroactive steroids. Some CNS disorders include components where the Peripheral Nervous System (“PNS”) is also compromised. Exemplary CNS disorders include multiple sclerosis, spinal muscular atrophy (believed to be due to loss of function of neuronal cells in the anterior horn of the spinal cord), muscle relaxation in spinal spasticity, cerebral palsy, trigeminal neuralgia, migraine, Alzheimer's disease, Huntington's chorea, Parkinson's disease, Creutzfeldt-Jakob's disease, Friedreich disease, retinal degenerations and photo-induced damage to the retina including photoretinitis, retinitis pigmentosa, age-related macular degeneration (AMD) and macular degeneration, delirium, dementia and amnestic and other cognitive disorders (delirium; dementia, such as dementia of Alzheimer's type, vascular dementia, dementia due to HIV disease, dementia due to head trauma, dementia due to Parkinson's disease, dementia due to Huntington's disease, dementia due to Pick's disease, dementia due to Creutzfeldt-Jakob disease, dementia due to general medical condition, substance-induced dementia, dementia due to multiple etiologies, dementia NOS (hereinafter “not otherwise specified” is abbreviated NOS); amnestic disorders, (such as amnestic disorder due to general medical condition, substance-induced amnestic disorder, anmestic disorder NOS: cognitive disorder NOS); ischemic or hemorrhagic cerebral vascular incidents including stroke and traumatic brain injury (TBI), phakomatoses (particularly neurofibromatosis), amyotrophic lateral sclerosis, schizophrenia, mood disorders (such as depressive disorder, including major depressive disorder-single episode or recurrent, dysthymic disorder, depressive disorder NOS; bipolar disorder, including, bipolar I disorder, bipolar II disorder, cyclothymic disorder, bipolar disorder NOS, mood disorder due to general medical condition, substance-induced mood disorder, mood disorder NOS), drug withdrawal symptoms, stuttering, autism, autism spectrum disorders, and convulsive disorders such as epilepsy. CNS disorders also includes mental disorders described in the American Psychiatric Association's Diagnostic and Statistical Manual of Mental Disorders, 5th edition (DSM-V) and include anxiety disorders (panic disorder without agoraphobia, panic disorder with agoraphobia, agoraphobia without history of panic disorder, specific phobia, social phobia, obsessive compulsive disorder, posttraumatic stress disorder, acute stress disorder, generalized anxiety disorder, social anxiety disorder, anxiety disorder due to a medical condition, substance induced anxiety disorder, anxiety disorder not otherwise specified (NOS)), mood disorders, sleep disorders (primary sleep disorder, e.g. primary insomnia, primary hypersomnia, narcolepsy, breathing-related sleep disorder, circadian rhythm sleep disorder, dysomnia NOS: parasomnia, including, nightmare disorder, sleep terror disorder, sleepwalking disorder, parasomnia NOS; sleep disorder secondary to another mental disorder, e.g. sleep disorder secondary to anxiety, mood disorder and/or other mental disorder: sleep disorder due to general medical condition and substance-induced sleep disorder), attention deficit, attention deficit hyperactivity, and disruptive behavior disorders (attention deficit/hyperactivity disorder—combined type, predominantly inattentive type and predominantly hyperactive-impulsive type: attention deficit/hyperactivity disorder NOS; conduct disorder, oppositional defiant disorder and disruptive behavior disorder NOS) and substance related disorders. Mental disorders also include eating disorders such as anorexia and bulimia. Further mental disorders and criteria for those disorders are described in the American Psychiatric Association's Diagnostic and Statistical Manual of Mental Disorders, 5.sup.th edition (DSM-V), the contents of which are hereby incorporated by reference in their entirety. Also included in are rare diseases and pediatric epilepsies other neurosteroid deficiency diseases including Rett Syndrome, Angelman Syndrome, Infantile Spasms, CDKL5 epilepsy, Postpartum depression, Tremor, Fragile X syndrome, Dravet's syndrome, Prader Willi, 15q11-q13 duplication deletion syndrome, Autoimmune epileptic encephalopathies, Lennox Gastaut Syndrome, Childhood Absence Epilepsy, Catamenial epilepsy, Status epilepticus, Suicidal ideation, Other genetic epilepsy syndromes, Seizures, Traumatic Brain Injury, Ischemic stroke, Spinal cord injury, Premenstrual dysphoric disorder, Chronic pain, and/or Migraine.

As used herein, a “PNS disorder” is a disease or condition of the peripheral nervous system that can be treated, prevented, managed or ameliorated with a compound or composition provided herein. Certain PNS disorders are responsive to increasing endogenous neuroactive steroids. Some PNS disorders involve motor nerve and/or sensory nerve dysfunction and can include components where the spinal cord and/or the brain are also compromised. Exemplary PNS disorders include neuropathic disorders (neuropathic disorders include neuropathies associated with a metabolic disturbance such as diabetic neuropathy, drug-induced neuropathies such as alcohol induced neuropathy and vincristine-induced neuropathy, neuropathies associated with an inflammatory process as in Guillain-Barre syndrome, chronic inflammatory demyelinating polyneuropathy, neuropathies associated with enzyme deficiency as in Fabry's disease and Krabbe's disease, peripheral neuropathy, infectious neuropathic conditions such as post-herpetic and HIV-induced neuralgia, hereditary motor and sensory neuropathies such as Charcot-Marie-Tooth disease), and radiculoneuropathic diseases.

As used herein, a “neurodegenerative process” is characterized by the dysfunction and death of the neurons leading to the loss of the neurological functions mediated by the brain (CNS), the spinal cord and the PNS. They can result, amongst others, from pathological situations known collectively under the term of neurodegenerative diseases or affections, traumatism, or exposure to toxins.

As used herein, a “neuroprotective property” is the ability of a compound of the invention to treat a neurodegenerative process.

As used herein and unless otherwise indicated, the terms “neurosteroids” and “neuroactive steroids” refer to steroids naturally produced in a subject and alter neuronal excitability through interaction with ligand-gated ion channels and other cell surface receptors. Neurosteroids are produced in the brain. Neuroactive steroids are produced by conversion of peripherally-derived adrenal steroids or gonadal steroids. Examples of neurosteroids and neuroactive steroids are: pregnenolone, pregnanolone, allopregnanolone, tetrahydrodeoxycorticosterone, dehydroepiandrosterone and progesterone. Neuroactive steroids can have effects in the CNS and peripherally.

The term “treat” as used herein means decrease, reverse, suppress, attenuate, diminish, arrest, or stabilize the development or progression of a disease (including., a disease or disorder delineated herein), lessen the severity of the disease or improve the symptoms associated with the disease. In one aspect, treatment does not include prevention.

“Disease” means any condition or disorder that damages or interferes with the normal function of a cell, tissue, or organ.

As used herein “subject” is an animal, typically a mammal, including human, such as a patient.

As used herein, and unless otherwise specified, the terms “therapeutically effective amount” and “effective amount” of a compound refer to an amount sufficient to provide a therapeutic benefit in the treatment, prevention and/or management of a disease, to delay or minimize one or more symptoms associated with the disease or disorder to be treated. The terms “therapeutically effective amount” and “effective amount” can encompass an amount that improves overall therapy, reduces or avoids symptoms, or causes of disease or disorder, or enhances the therapeutic efficacy of another therapeutic agent.

The terms “co-administration” and “in combination with” include the administration of two therapeutic agents (for example, compounds of this invention and lorazepam) either simultaneously, concurrently or sequentially with no specific time limits. In one embodiment, both agents are present in a subject at the same time or exert their biological or therapeutic effect at the same time. In one embodiment, the two therapeutic agents are in the same composition or unit dosage form. In another embodiment, the two therapeutic agents are in separate compositions or unit dosage forms.

It will be recognized that some variation of natural isotopic abundance occurs in a synthesized compound depending upon the origin of chemical materials used in the synthesis. Thus, a preparation of etifoxine will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance. Wada, E et al., Seikagaku, 1994, 66:15; Gannes, L Z et al., Comp Biochem Physiol Mol Integr Physiol, 1998, 119:725.

In the compounds of this invention, any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also, unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope. In some embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6533 (98% deuterium incorporation), at least 6600 (99% deuterium incorporation), or at least 6633.3 (99.5% deuterium incorporation).

The term “isotopologue” refers to a species that differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above, the relative amount of such isotopologues in total will be less than 49.9% of the compound. In other embodiments, the relative amount of such isotopologues in total will be less than 47.5%, less than 40%, less than 32.5%, less than 25%, less than 17.5%, less than 10%, less than 5%, less than 3%, less than 1%, or less than 0.5/0 of the compound.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient. Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, .beta.-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid. Standard methods for the preparation of pharmaceutically acceptable salts and their formulations are well known in the art, and are disclosed in various references, including for example, “Remington: The Science and Practice of Pharmacy”. A. Gennaro, ed., 20th edition, Lippincott, Williams & Wilkins, Philadelphia, Pa.

The compounds of the present invention (including, compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers, or less than “X”% of other stereoisomers (wherein X is a number between 0 and 100, inclusive) are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound

The term “stable compounds,” as used herein, refers to compounds which possess stability sufficient to allow for their manufacture and which maintain the integrity of the compound for a sufficient period of time to be useful for the purposes detailed herein (including, formulation into therapeutic products, intermediates for use in production of therapeutic compounds, isolatable or storable intermediate compounds, treating a disease or condition responsive to therapeutic agents).

“D” and “d” both refer to deuterium. Unless otherwise indicated, “stereoisomer” refers to both enantiomers and diastereomers.

The term “optionally substituted with deuterium” means that one or more hydrogen atoms in the referenced moiety may be replaced with a corresponding number of deuterium atoms.

The present invention includes prodrugs of the compounds of Formula I above. In general, such prodrugs will be functional derivatives of the compounds of Formula I that are readily convertible in vivo into the required compound of Formula I. Conventional procedures for the selection and preparation of suitable prodrug derivatives are described, for example, in Design of Prodrugs, ed. H. Bundgaard. Elsevier, 1985. Such prodrugs include but are not limited to ester prodrugs from alcohols and acids and phosphate prodrugs of alcohols. The prodrug can be formulation to achieve a goal of improved chemical stability, improved patient acceptance and compliance, improved bioavailability, prolonged duration of action, improved organ selectivity, improved formulation (including, increased hydrosolubility), and/or decreased side effects (including, toxicity).

Where the compounds of the present invention have at least one asymmetric center, they may accordingly exist as enantiomers. Where the compounds possess two or more asymmetric centers, they may additionally exist as diastereoisomers. Specifically, etifoxine exists as a racemic mixture and R-Etifoxine and S-Etifoxine have been prepared. U.S. Pat. No. 8,110,569. The present invention includes deuterated analogs of R-Etifoxine and deuterated analogs of S-Etifoxine. It is to be understood that all such stereoisomers and mixtures thereof in any proportion are encompassed within the scope of the present invention. Where the compounds possess geometrical isomers, all such isomers and mixtures thereof in any proportion are encompassed within the scope of the present invention. Tautomers of the compounds of the invention are encompassed by the present application. Thus, for example, a carbonyl includes its enol tautomer.

As used herein “pure S-etifoxine” are deuterated analogs that are is substantially free from deuterated R-etifoxine analogs (i.e., in enantiomeric excess). In other words, the “S” form of the deuterated etifoxine is substantially free from the “R” form of the compound and is, thus, in enantiomeric excess of the “R” form.

The term “enantiomerically pure” or “pure enantiomer” denotes that the compound comprises more than 75% by weight, more than 80% by weight, more than 85% by weight, more than 90% by weight, more than 91% by weight, more than 92% 9 by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of the deuterated etifoxine analog.

As used herein and unless otherwise indicated, the term “enantiomerically pure R-etifoxine” refers to the deuterated analog that at least about 80% by weight deuterated R-etifoxine and at most about 20% by weight deuterated S-etifoxine, at least about 90% by weight deuterated R-etifoxine and at most about 10% by weight deuterated S-etifoxine, at least about 95% by weight deuterated R-etifoxine and at most about 5% by weight deuterated S-etifoxine, at least about 99% by weight deuterated R-etifoxine and at most about 1% by weight deuterated S-etifoxine, at least about 99.9% by weight deuterated R-etifoxine or at most about 0.1% by weight deuterated S-etifoxine. In certain embodiments, the weights are based upon total weight of deuterated etifoxine analog.

As used herein and unless otherwise indicated, the term “enantiomerically pure S-etifoxine” refers to at least about 80% by weight deuterated S-etifoxine and at most about 20% by weight deuterated R-etifoxine, at least about 90% by weight deuterated S-etifoxine and at most about 10% by weight deuterated R-etifoxine, at least about 95% by weight deuterated S-etifoxine and at most about 5% by weight deuterated R-etifoxine, at least about 99% by weight deuterated S-etifoxine and at most about 1% by weight deuterated R-etifoxine or at least about 99.9% by weight deuterated S-etifoxine and at most about 0.1% by weight deuterated R-etifoxine. In certain embodiments, the weights are based upon total weight of deuterated etifoxine analog.

The term “AUC₀₋₁₂” as used herein means area under the concentration-time curve from time 0 to time 12 hours.

The term “AUC₀₋₂₄” as used herein means area under the concentration-time curve from time 0 to time 24 hours.

The term “AUCₗₐₛₜ” as used herein means area under the concentration-time curve from time 0 to the time of last quantifiable concentration (tₗₐₛₜ), calculated using the linear trapezoidal rule for increasing concentrations and the logarithmic rule for decreasing concentrations.

The term “AUC_{inf}” as used herein means area under the concentration-time curve from time 0 extrapolated to infinity.

The term “Cₘₐₓ” as used herein means maximum observed plasma concentration.

The term “Cₘᵢₙ” as used herein means minimum observed means pre-dose observed plasma concentration (over the initial dosing interval (Day 7)).

The term “ARCₘₐₓ” as used herein means Cₘₐₓ Accumulation Ratio: Cₘₐₓ (Day 7) divided by Cₘₐₓ (Day 1).

The term “ARAUC₀₋₁₂” as used herein means AUC₀₋₁₂ Accumulation Ratio: AUC₀₋₁₂ (Day 7) divided by AUC₀₋₁₂ (Day 1).

The term “PK” as used herein means pharmacokinetic.

The term “q12h” as used herein means every 12 hours.

The term “Tₘₐₓ” as used herein means time of maximum observed plasma concentration.

The term “t_{1/2}” as used herein means the apparent plasma terminal elimination half-life.

### Multi-Day Therapeutic Use of Etifoxine

The hydrochloride salt of etifoxine [6-chloro-2-(ethylamino)-4-methyl-4-phenyl-4H-3,1-benzoxazine] known as Stresam™ has been known to exhibit metabolic induction, whereby dosing over time results in an induction of metabolism in humans. Deuterated etifoxine for treatment of anxiety was previously disclosed in U.S. Pat. No. 10,736,901, entitled “Deuterated Analogs of Etifoxine, Their Derivatives and Uses Thereof,” to Olivier Dasse. The '901 patent discloses various deuterated analogs of etifoxine and methods of making the same, and is incorporated herein by reference for its teachings regarding deuterated analogs of etifoxine and methods of making the same. The present inventors fully expected deuterated etifoxine to exhibit a metabolic induction effect similar to etifoxine.

During multi-day human clinical trials involving deuterated etifoxine, the present inventors confirmed that deuterated etifoxine exhibited a metabolic autoinduction effect similar to etifoxine at doses greater than 100 mg bid. However, the present inventors surprisingly discovered that the metabolic induction in deuterated etifoxine differs from what was known about the metabolic induction effect for etifoxine (non-deuterated). Namely, deuterated etifoxine does not exhibit metabolic induction when administered within the therapeutic dosing range (100 mg bid). Based on this surprising discovery, the present inventors have developed novel dosage regimens for deuterated etifoxine that are useful in the treatment of chronic conditions, such as chronic anxiety.

The present inventors have found that deuterated etifoxine administered at a dosage of 150 mg bid (twice daily) induces metabolism. The examples below show that administering deuterated etifoxine at a dose of 100 mg bid did not induce metabolism. Administering deuterated etifoxine at doses which do not induce metabolism are contemplated. Such doses may include any dose that is less than 150 mg bid, and preferably includes any dose such as 145 mg bid, 140 mg bid, 135 mg bid, 130 mg bid, 125 mg bid, 120 mg bid, 115 mg bid, 110 mg bid, 105 mg bid, 100 mg bid, 95 mg bid, 90 mg bid, 85 mg bid, 80 mg bid, 75 mg bid, 70 mg bid, 65 mg bid, 60 mg bid, 55 mg bid, 50 mg bid, 45 mg bid, 40 mg bid, 35 mg bid, 30 mg bid, 25 mg bid, 20 mg bid, 15 mg bid, 10 mg bid or less may be administered.

The present inventors expect that the same total daily amount of deuterated etifoxine of 300 qd would be equivalent to 150 mg bid for inducing metabolism. Accordingly, qd regimens equivalent to 200 qd have been shown to avoid inducing metabolism. The inventors contemplate that doses of 200 mg qd, 190 mg qd, 180 mg qd, 170 mg qd 160 mg qd, 150 mg qd, 140 mg qd, 130 mg qd, 120 mg qd, 110 mg qd, 100 mg qd, 90 mg qd, 80 mg qd, 70 mg qd, 60 mg qd, 60 mg qd, 50 mg qd, 40 mg qd, 30 mg qd 20 mg qd may be administered in a manner that avoids inducing metabolism.

The ability to administer deuterated etifoxine in a manner that avoids autoinduction of metabolism may advantageously provide dosing regimens that are lower than would have otherwise been needed. The present inventors contemplate dosing regimens for treating anxiety on a chronic basis that include administering deuterated etifoxine at 100 mg qd, and preferably lower. For example, a dosing regimen for treating chronic anxiety may include 100 mg qd, 90 mg qd, 80 mg qd, 70 mg qd, 60 mg qd, or lower.

Other indications may be treated using deuterated etifoxine in a manner that avoids autoinduction at different doses and dosing frequencies.

In addition to racemic deuterated etifoxine, the present inventors contemplate the use of deuterated S-etifoxine, deuterated R-etifoxine with any of the above methods.

### Metabolic Differences Between Deuterated Etifoxine and Etifoxine

The present inventors studied the comparative stability of deuterated etifoxine and etifoxine in human, rat, and mouse liver microsomes:

TABLE 1
Deuterated Etifoxine and Etifoxine Stability
in Human, Rat, and Mouse Liver Microsomes

| | |
|---|---|
| T½ (minutes) | Clᵢₙₜ (μL/min/mg) |

| | Deuterated | | Deuterated | |
|---|---|---|---|---|
| Species | Etifoxine | Etifoxine | Etifoxine | Etifoxine |
| Human | 41 | 22 | 170.2 | 308.7 |
| Rat | 14 | 8 | 503.3 | 915.1 |
| Mouse | 15 | 11 | 463.5 | 656.9 |

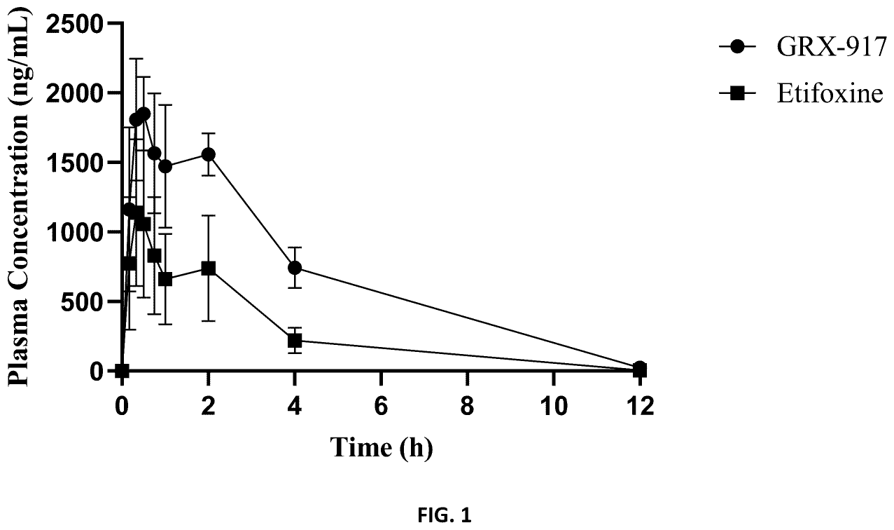

Deuterated etifoxine is metabolically more stable (lower intrinsic clearance (Clint) and longer half-life) than etifoxine in human, rat, and mouse liver microsomes (Table 1). As a result of this greater metabolic stability, GRX-917 is expected to have better oral bioavailability than etifoxine. In addition, the ratio of GRX-917 to M4 following a dose of GRX-917 is expected to be higher than the ratio of etifoxine to M4 following a comparable dose of etifoxine. The major metabolite (M4) is an enzyme inducer and contributes to the in vivo autoinduction. In rats, this improved metabolic stability translates into an approximately 3-fold greater AUC for GRX-917 as compared to etifoxine as shown in FIG. 1.

### Multiple Ascending Dosing Study with Deuterated Etifoxine in Healthy Subjects

This was a Phase 1, single-center, prospective, randomized, double-blind, placebo-controlled study of multiple-ascending doses (MAD) of deuterated etifoxine in racemic form administered orally to healthy adult male and female subjects.

The first dose of study drug was given on Day 1. On Days 1 through 6, subjects subsequently received two (q12h) oral doses of study drug each day. A final single dose of study drug was given on Day 7. Each dose of study drug was given with a 240 mL glass of water at the end of ingestion of a standard meal. The meal was provided 30 minutes before dosing and was completed within 30 minutes or less. On Day 1 and Day 7, subjects were required to fast for 8 hours before the morning meal dose and for 2 hours post dose (water was freely available during this time).

Fifty-eight (58) subjects were enrolled in the MAD portion of study (43 subjects on deuterated etifoxine and 15 subjects on placebo). All of the available data for the 43 subjects treated with deuterated etifoxine were included in the PK Population.

### Deuterated Etifoxine—Multiple Oral Dose Pharmacokinetics on Day 7

The geometric mean (CV %) trough concentrations (12 hours post-last dose) of deuterated etifoxine on Days 2, 4, 5, 6 and 7 following q12h (bid) dosing are presented in Table 2.

TABLE 2
Geometric Mean (% CV) Trough Concentrations
of Deuterated Etifoxine

| | 100 mg | | 150 mg | | 200 mg | | 300 mg | |
|---|---|---|---|---|---|---|---|---|
| | q12 h | | q12 h | | q12 h | | q12 h | |
| | | % | | % | | % | | % |
| Day | Mean | CV | Mean | CV | Mean | CV | Mean | CV |
| 1 | 11.6 | 41.6 | 28.1 | 36.2 | 42.8 | 56.5 | 54.9 | 26.7 |
| 2 | 36.1 | 52.4 | 43.8 | 25.1 | 72.3 | 47.5 | 87.4 | 29.2 |
| 4 | 39.3 | 57.4 | 44.5 | 51.0 | 67.2 | 45.9 | 71.0 | 25.4 |
| 5 | 31.9 | 43.8 | 39.2 | 58.6 | 62.4 | 45.4 | 70.0 | 23.6 |
| 6 | 34.0 | 36.0 | 37.7 | 54.2 | 53.7 | 59.5 | 66.3 | 28.7 |
| 7 (pre- | 27.7 | 28.8 | 33.3 | 49.4 | 58.9 | 46.4 | 63.0 | 34.8 |
| dose) | | | | | | | | |
| 7 (12-hr | 27.1 | 34.9 | 31.4 | 48.6 | 51.4 | 54.3 | 60.4 | 26.8 |
| post-dose) | | | | | | | | |

At each dose level, geometric mean trough deuterated etifoxine concentrations increased until Day 4 at 100 and 150 mg q12h and until Day 2 at 200 and 300 mg q12h. Subsequently, trough concentrations decreased until 12-hours post-last dose on Day 7, indicating a possible autoinduction effect.

The geometric mean (CV %) PK parameters of deuterated etifoxine on Day 7 following q12h (bid) dosing for 7 days are presented in Table 3.

TABLE 3
Geometric Mean (% CV) Summary of the Plasma Pharmacokinetic
Parameters for Deuterated Etifoxine on Day 7

| | 100 mg q12 h | | | 150 mg q12 h | | | 200 mg q12 h | | | 300 mg q12 h | | |
|---|---|---|---|---|---|---|---|---|---|---|---|---|
| ARCₘₐₓ | 9 | 1.04 | 37.3 | 8 | 0.608 | 28.8 | 13 | 0.711 | 42.9 | 9 | 0.551 | 25.9 |
| ARAUC₀₋₁₂ | 9 | 1.31 | 23.6 | 7 | 0.729 | 37.9 | 11 | 0.863 | 19.5 | 8 | 0.737 | 21.9 |

^{a}Median(range)

As the criteria for reporting of half-life values required the terminal phase to span at least 2 half-lives (i.e., span >2), only 1/9, 3/8, 3/13 and 0/9 subjects at the 100, 150, 200 and 300 mg q12h dose regimens had half-lives that could be accurately determined. At 100 mg q12h, both geometric mean accumulation ratios of deuterated etifoxine (ARCmax and AUC0-12) were 1.04 and 1.31, respectively, suggesting minimal accumulation upon multiple dosing for 7 days and no autoinduction. However, geometric mean ARCmax and ARAUC0-12 decreased to less than unity at doses greater than 100 mg q12h and generally decreased as the dose was increased to 0.551 and 0.737 at 300 mg q12h, respectively, which suggests an autoinduction effect.

Over the dose range on Day 7, AUC0-12 and Cmax increased in a dose proportional manner. Statistical analysis of dose proportionality confirmed dose proportionality with slope estimates (90% CI) of 0.86 (0.66, 1.07) and 0.91 (0.67, 1.14) for Cmax and AUC0-12, respectively. Statistical analysis indicated that Tmax was independent of dose on Day 7.

### Time-Dependent Kinetics

Deuterated etifoxine did not exhibit decreased exposure when dosed for 7 days at 100 mg BID, however, ARCmax and ARAUC0-12 decreased below 1 at doses greater than 100 mg BID, indicating an autoinduction effect.

### Renal Elimination

Deuterated etifoxine demonstrated low renal clearance. The fraction of dose excreted as deuterated etifoxine was <0.004%.

### Conclusions

Following single and multiple oral doses of 100-300 mg q12h, deuterated etifoxine was rapidly absorbed with median Tmax ranging from 2.00 to 3.52 h post-dose.

Plasma deuterated etifoxine concentrations declined exponentially in an apparent overall biphasic manner on Day 7 with a geometric mean t1/2 ranging between 38.8 and 82.1 h.

Deuterated etifoxine Cmax and AUC0-12 increased dose proportionally on Day 7.

Deuterated etifoxine accumulation upon multiple dosing for 7 days was minimal at 100 mg q12h with ARCmax and ARAUC0-12 values of 1.04 and 1.31, respectively; however, these values decreased to between 0.551 to 0.863 at doses greater than 100 mg BID, indicating an autoinduction effect.

### Example 1

Deuterated etifoxine is administered to human patients in an amount of 100 mg BID (twice daily) for the treatment of anxiety over a period of at least 7 days. The lack of autoinduction of deuterated etifoxine metabolism is demonstrated by an AUCmax and ARAUC₀₋₁₂ of equal or greater than one for a period of at least 7 days.

### Example 2

Deuterated etifoxine is administered to human patients in an amount of 100 mg QD (once daily) for the treatment of anxiety over a period of at least 7 days. The lack of autoinduction of deuterated etifoxine metabolism is demonstrated by an AUCmax and ARAUC₀₋₁₂ of equal or greater than one for a period of at least 7 days.

### Example 3

Deuterated etifoxine is administered to human patients in an amount of 60mg QD (once daily) for the treatment of anxiety over a period of at least 7 days. The lack of autoinduction of deuterated etifoxine metabolism is demonstrated by an AUCmax and ARAUC₀₋₁₂ of equal or greater than one for a period of at least 7 days.

### Example 4

Deuterated etifoxine is administered to human patients in an amount of 50 mg QD (once daily) for the treatment of anxiety over a period of at least 7 days. The lack of autoinduction of deuterated etifoxine metabolism is demonstrated by an AUCmax and ARAUC₀₋₁₂ of equal or greater than one for a period of at least 7 days.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

1. A method for treating disease or disorder in a subject in need thereof, comprising orally administering to said subject a compound, wherein the compound is a compound of formula (I): 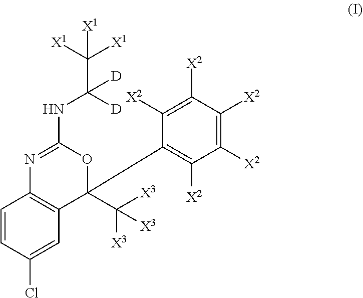
including pharmaceutically acceptable salts, solvates, and prodrugs thereof, wherein each X¹, X², X³ are independently selected from hydrogen or deuterium, wherein said compound is administered at a dose and frequency that is effective for treatment of anxiety and that exhibits an ARCₘₐₓ and ARAUC₀₋₁₂ over a period of 7 days that is equal to or greater than 1.0.

2. The method of claim 1, wherein ARCₘₐₓ over a period of 7 days is the ratio of Cmax at day 7 divided by Cmax at day 1.

3. The method of claim 1, wherein the dosing is BID or QD.

4. The method of claim 1, wherein the dose is 100 mg or less.

5. The method of claim 1, wherein the dose ranges from 20 mg to 100 mg.

6. The method of claim 1, wherein the dosage is 60 mg administered QD.

7. The method of claim 1, wherein the disease or disorder is selected from panic disorder without agoraphobia, panic disorder with agoraphobia, agoraphobia without history of panic disorder, specific phobia, social phobia, obsessive compulsive disorder, posttraumatic stress disorder, acute stress disorder, generalized anxiety disorder, anxiety disorder due to a medical condition, substance induced anxiety disorder, or anxiety with somatic manifestations.

8. The method of claim 1, wherein the compound is 6-chloro-N-(ethyl-d5)-4-methyl-4-phenyl-4H-3,I-benzoxazin-2-amine, or a pharmaceutically acceptable salt thereof.

9. The method of claim 1, wherein the compound includes deuterium in an abundance that is at least 3340 times greater than the natural abundance of deuterium.

10. The method of claim 1, wherein the compound is a racemate.

11. A method for treating a disease or disorder in a subject in need thereof, comprising orally administering to said subject 6-chloro-N-(ethyl-d5)-4-methyl-4-phenyl-4H-3,I-benzoxazin-2-amine, wherein said compound is administered at a dose and frequency that is effective for treatment of anxiety and that exhibits an ARCₘₐₓ and ARAUC₀₋₁₂ over a period of 7 days that is equal to or greater than 1.0.

12. The method of claim 11, wherein the dosing is BID or QD.

13. The method of claim 11, wherein the dosing is QD.

14. The method of claim 11, wherein the dose is 100 mg or less.

15. The method of claim 11, wherein the dose ranges from 50 mg to 100 mg.

16. The method of claim 11, wherein the dosage is 60 mg administered QD.

17. The method of claim 11, wherein the disease or disorder is selected from panic disorder without agoraphobia, panic disorder with agoraphobia, agoraphobia without history of panic disorder, specific phobia, social phobia, obsessive compulsive disorder, posttraumatic stress disorder, acute stress disorder, generalized anxiety disorder, anxiety disorder due to a medical condition, substance induced anxiety disorder, or anxiety with somatic manifestations.

18. The method of claim 11, wherein the compound is 6-chloro-N-(ethyl-d5)-4-methyl-4-phenyl-4H-3,I-benzoxazin-2-amine, or a pharmaceutically acceptable salt thereof.

19. The method of claim 11, wherein the compound includes deuterium in an abundance that is at least 3340 times greater than the natural abundance of deuterium.

20. The method of claim 11, wherein the compound is a racemate.

21. A pharmaceutical composition comprising a compound of formula (I): 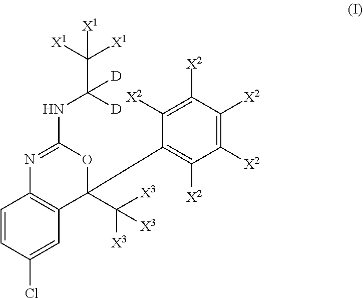
including pharmaceutically acceptable salts, solvates, and prodrugs thereof, wherein each X¹, X², X³ are independently selected from hydrogen or deuterium, and a pharmaceutically acceptable excipient, wherein the compound is present in an amount that when administered once daily exhibits an ARCₘₐₓ and ARAUC₀₋₁₂ over a period of 7 days that is equal to or greater than 1.0.

22. The pharmaceutical composition of claim 21, wherein the compound is 6-chloro-N-(ethyl-d5)-4-methyl-4-phenyl-4H-3,I-benzoxazin-2-amine, or a pharmaceutically acceptable salt thereof.

23. The pharmaceutical composition of claim 21, wherein the amount ranges from 20 mg to 100 mg.

24. The pharmaceutical composition of claim 21, wherein the amount is 60 mg administered QD.
